(19) United States
(12) Patent Application Publication
(10) Pub. No.: US 2023/0233069 A1
(43) Pub. Date: Jul. 27, 2023

(54) DEVICES, SYSTEMS, AND RELATED METHODS FOR VISUAL GUIDANCE OF TRANSESOPHAGEAL ECHOCARDIOGRAPHY PROBES

(71) Applicant: Haluk Un, Plymouth, MN (US)

(72) Inventor: Haluk Un, Plymouth, MN (US)

(21) Appl. No.: 18/101,605

(22) Filed: Jan. 26, 2023

Related U.S. Application Data
(60) Provisional application No. 63/303,504, filed on Jan. 27, 2022.

(51) Int. Cl. A61B 1/05; A61B 1/00; A61B 1/12; A61B 1/06

(52) U.S. Cl. CPC A61B 1/05; A61B 1/0004; A61B 1/0014; A61B 1/00082; A61B 1/00091; A61B 1/00117; A61B 1/126; A61B 1/00135; A61B 1/0676

(57) ABSTRACT

The present disclosure is directed to a device, system, and related methods for providing visual assistance to the placement of an endoscopic device acts as a cover for protecting both the probe and tube body of an endoscopic device form bacterial biofilm build-up during use. The device comprises a flexible sleeve, the sleeve being at least partially elastic, which is fitted over and encloses the tip of an endoscopic device such as a TEE probe, with the only opening in the sleeve tip being placed to expose the sensor apparatus of the probe. The tip of the sleeve comprises an image capture device which is coupled, via a wired connection that runs through the sleeve, to a control unit having a transceiver, such that captured image data can be transmitted to external devices, or an integrated display device, in real-time, without the transmission being obstructed by the patient’s body. The design of the sleeve also allows it to be used as a conduit for various other transmissions such as fluid and fibre optics.

### CROSS REFERENCE TO RELATED APPLICATIONS

This patent application claims the benefit of and priority of U.S. Provisional Application No. 63/303,504, filed 27 Jan. 2022.

### FIELD OF INVENTION

The present invention relates generally to the field of ultrasonography apparatus. More specifically, the present invention proposes a visual assistance device configured to provide visual support to the operator during an endoscopic procedure.

### BACKGROUND

An echocardiogram uses echocardiography to assess the structure and function of the heart. During the procedure, a transducer (like a microphone) sends out ultrasonic sound waves. When the transducer is placed at certain locations and angles, the ultrasonic sound waves move through the skin and other body tissues to the heart tissues, where the waves bounce or “echo” off of the heart structures. The transducer picks up the reflected waves and sends them to a computer. The computer displays the echoes as images of the heart walls and valves.

A traditional echocardiogram is done by putting the transducer on the surface of the chest. This is called a transthoracic echocardiogram. A more recent technique developed, the transesophageal echocardiogram (TEE), is done by inserting a probe with a transducer down the esophagus. This provides a clearer image of the heart because the sound waves do not have to pass through skin, muscle, or bone tissue. The TEE probe is much closer to the heart since the esophagus and heart are right next to each other, however correctly placing the TEE probe within the esophagus is difficult and has associated risks.

While the TEE probe is placed in the esophagus, it is advanced to the pharynx through the mouth, and then the esophagus is reached, bypassing the upper esophageal sphincter. The upper esophageal sphincter and trachea (larynx) are located side by side. The operator who performs the operation makes this distinction using only his feelings and experiences. The TEE process is done completely “blind.” Performing the procedure in this way causes difficulties and can be the source of various complications during the procedure.

Attempts have been made to address the aforementioned issues by the proposal of new camera-containing TEE probes to provide visualization during the procedure. These probes make it necessary to have an extra device inserted alongside the TEE probes, making insertion more difficult.

U.S. Pat. Publication No. US10925576B2, “Apparatus, system, and methods for proper transesophageal echocardiography probe positioning by using camera for ultrasound imaging,” describes adding a removable or detachable carrier containing a camera and illumination to a TEE probe, thereby allowing the user to view the placement of the probe and minimize or reduce the risk of esophageal and pharyngeal complications during the positioning of the TEE probe into the patient’s esophagus. Once the TEE probe has been properly positioned in the esophagus, the carrier is removed from the esophagus.

U.S. Pat. Publication No. US6884220B2 “Optical Transesophageal Echocardiography Probe,” describes an optical transesophageal echocardiography probe (OPTEE) having an optical fiber bundle, a suction channel and light channels for illumination, wherein the OPTEE has a circumference along its distal tip.

International Patent Publication No. WO2021116474A1 “Systems and methods for guiding an ultrasound probe,” describes an ultrasound device that includes a probe including a tube sized for insertion into a patient and an ultrasound transducer disposed at a distal end of the tube. A camera is mounted at the distal end of the tube in a fixed spatial relationship to the ultrasound transducer. At least one electronic processor is programmed to: control the ultrasound transducer and the camera to acquire ultrasound images and camera images respectively while the ultrasound transducer is disposed in vivo inside the patient.

In the solutions proposed by the available prior art, different devices are required for TEE probes of different brands and models. These devices are partially attached to the TEE probe, requiring the use of hard and thick material to establish a strong and solid connection. In this case, the overall thickness of the probe increases, the probe flexibility decreases, and the hard material used may damage living tissue.

Another problem associated with endoscopic procedures, such as the insertion of TEE probes, is the increased risk of contamination that comes from reusable probes. While endoscopic devices rest within the body, a layer of bacteria gradually builds on the shaft and senso, and if left unattended can create a biofilm. The biofilm layer demands immediate cleaning with expensive equipment to properly remove and disinfect after a probe has been used, and improper cleaning creates a risk point for bacterial transmission.

The solutions proposed in the available prior art fail to provide sufficient protective covering for the shafts of the endoscopic devices while they rest within a patient’s oesophagus. These problems necessitate a new, more universal design that can be retrofitted to various makes and sizes of TEE probe while providing improved functionality in various ways.

It is within this context that the present invention is provided.

### SUMMARY

The present disclosure is directed to a device, system, and related methods for providing visual assistance to the placement of an endoscopic device acts as a cover for protecting both the probe and tube body of an endoscopic device form bacterial biofilm build-up during use. The device comprises an flexible sleeve, the sleeve being at least partially elastic, which is fitted over and encloses the tip of an endoscopic device such as a TEE probe, with the only opening in the sleeve tip being placed to expose the sensor apparatus of the probe. The tip of the sleeve comprises an image capture device which is coupled, via a wired connection that runs through the sleeve, to a control unit having a transceiver, such that captured image data can be transmitted to external devices, or an integrated display device, in real-time, without the transmission being obstructed by the patient’s body. The design of the sleeve also allows it to be used as a conduit for various other transmissions such as fluid and fiber optics.

According to a first aspect of the present disclosure, there is provided a Visual Assistance Device (VAD), comprising: an flexible sleeve, the sleeve being at least partially elastic, having a first open end for inserting an endoscopic device and a second closed end for encasing the tip of an endoscopic device, the second end comprising an opening for exposing a sensor of an endoscopic device housed therein; an image capture device disposed at the tip the second end of the flexible sleeve and being oriented coaxially with the sleeve so as to capture images of a region in front of the second end of the sleeve; one or more lighting elements positioned adjacent to the image capture device and being configured to illuminate an area about the image capture device.

The VAD further comprises a control unit comprising a transceiver, the control unit being configured to receive image data from the image capture device and transmit the image data to one or more external devices, the control unit being positioned outside of the sleeve with a wired connection for image data transmission extending along the entire length of the sleeve interior from the image capture device to the control unit.

In some embodiments, the flexible sleeve is formed of two or more layers of flexible material, including a first base layer that forms the interior wall and one or more secondary layers.

The one or more secondary layers may be arranged to form one or more channels on the base layer.

In such embodiments, the one or more channels may be sealed to facilitate their use as a fluid conduit.

In further embodiments, the image capture device and the one or more lighting elements are secured on a flexible strip which can be inserted through and removed from one of the channels to position the image capture unit in a desired position.

In yet further embodiments, the one or more of the channels have fluidic pipes threaded through them, the pipe being coupled at the open end of the sleeve to an injector system. The opposing opening of the one or more of the fluidic pipes may also comprise a nozzle directed at the image capture unit and is thereby configured to spray clean the image capture unit while the VAD is in use.

The VAD may further comprise an inflatable balloon membrane positioned at the second end of the sleeve.

Additionally, the one or more channels may house one or more fibre optic lines coupling the lighting elements to an external light source.

In some embodiments, the VAD further comprises a transparent protective cover arranged to at least partially cover the image capture device and lighting elements. The protective cover may be shaped into a pointed tip and oriented in a desired direction.

In some embodiments, the control unit is configured to receive and transmit captured Image data in real-time Alternatively, the control unit may be configured to receive and transmit captured Image data with a predetermined time delay.

In some embodiments, the control unit comprises a gripping mechanism for securing itself to the shaft of an endoscopic device.

In some embodiments, the control unit comprises an interface for adjusting one or more settings of the image capture device and the one or more lighting elements.

In some embodiments, the VAD further comprises a display device configured to communicate with the control unit to receive and display the captured image data.

There may be a wired connection between the control unit and the display device. The display device may be configured to perform one or more image processing operations on the received image data. The display device may comprise a touchscreen interface and be configured to instruct the control unit to perform one or more operations.

In some embodiments, the opening at the second end of the flexible sleeve is covered by a material configured to transmit ultrasonic waves.

According to another aspect of the present disclosure, there is provided an anti-infection cover for a medical probe, the cover comprising a flexible sleeve, the sleeve being at least partially elastic, having a first open end for inserting a medical probe and a second closed end for encasing the tip of a medical probe, the second end comprising an opening for exposing a sensor of a medical probe housed therein.

The cover may be provided with an antibacterial coating and may completely enclose the shaft of the medical probe installed therein to prevent transmission of infections.

### BRIEF DESCRIPTION OF THE DRAWINGS

Various embodiments of the invention are disclosed in the following detailed description and accompanying drawings.

FIG. 1-A illustrates top and front views of an example configuration of a Transesophageal Echocardiography (TEE) probe.

FIG. 1-B illustrates top and front views of an example configuration of a Visual Assist Device (VAD) according to the present disclosure.

FIG. 1-C illustrates top and front views of the example configuration of the VAD placed on the TEE probe.

FIG. 2-A illustrates a top view of the example VAD.

FIG. 2-B illustrates an anterior view of the tip of the example VAD.

FIGS. 2-C and D illustrate cross-sectional side views of the tip of the example VAD.

FIG. 3-A illustrates a side view of the tip of the example VAD.

FIG. 3-B illustrates a front view of the tip of the example VAD.

FIG. 3-C illustrates a top view of the tip of the example VAD.

FIG. 4-A illustrates a side view of the example VAD with a cross-sectional line.

FIG. 4-B illustrates a top view of the cross-section of the example VAD.

FIG. 5-A illustrates a front view of the camera of the example VAD.

FIG. 5-B illustrates a side view of the camera of the example VAD.

FIG. 6-A illustrates a view of the example VAD ready for placement on the TEE probe.

FIG. 6-B illustrates a view of the device placed on the TEE probe to provide wireless image transmission.

FIG. 6-C illustrates a screen display for showing images taken by the VAD.

FIG. 7 illustrates a view of the example VAD and wireless transmission device installed on the TEE probe and ready-to-use.

FIG. 8 illustrates a perspective isometric view of the example VAD configuration.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. Numerical quantities given in this description are approximate unless stated otherwise, meaning that the term “about” or “approximately” can be inferred when not expressly stated.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments.

Spatially relative terms, such as “under,” “below,” “lower,” “over,” “upper” and the like, may be used herein for ease of description to describe one element or feature’s relationship to another when the apparatus is right side up.

The terms “first,” “second,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

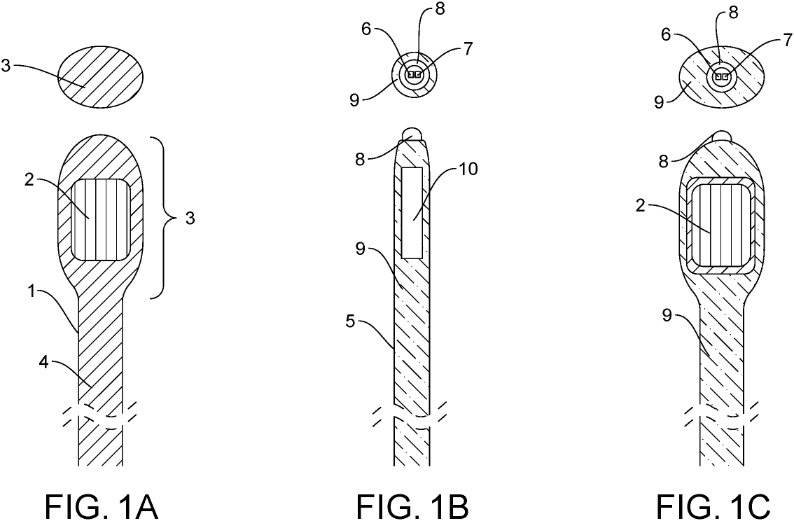

Referring to FIG. 1-A, a distal end of an example TEE probe (1) is shown with a standard configuration. The probe comprises a sensor (2) positioned on the side of a tip (3) section that has a larger diameter than the main body of the probe. The “distal end” as referred to herein refers to the part that is closer to the patient and far from the operator performing the operation. The ultrasonic sensor (2) is located on one side of the tip (3) of the TEE probe (1). This sensor (2) can be used to provide a detailed ultrasonic image of the heart using methods well known in the art. The part where the sensor (2) is located is slightly thicker in diameter than the shaft (4) of the TEE probe (1). The first 10-15 cm of the TEE probe (1), which has the sensor (2), can be manipulated by the operator and can be moved in the right, left, up, and down directions. As the TEE probe (1) is advanced into the patient’s esophagus, this part advances first and is manipulated and advanced towards the patient’s digestive system.

The VAD (5), seen in FIG. 1-B, is a visual assist device that includes a camera (6) and a light source (7) system, which can be placed on the TEE probe (1) and moves together with the probe (1). This device may include an image processing system and is configured to attach to the TEE probe (1) by encasing it in cover (9). The image acquisition system consists of the camera (6) and the light source (7) system.

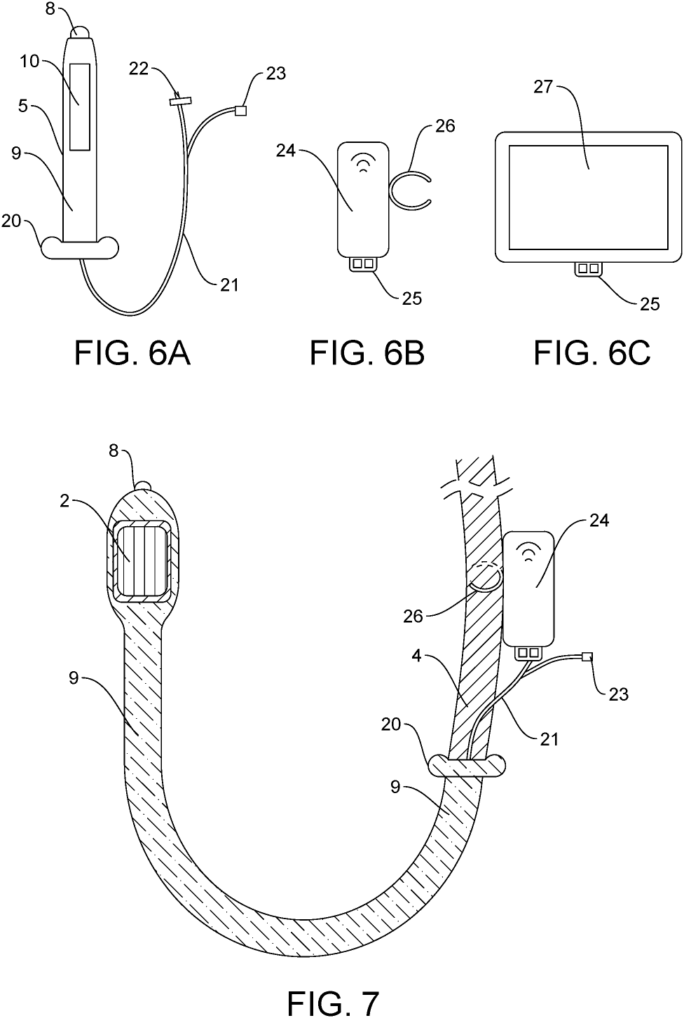

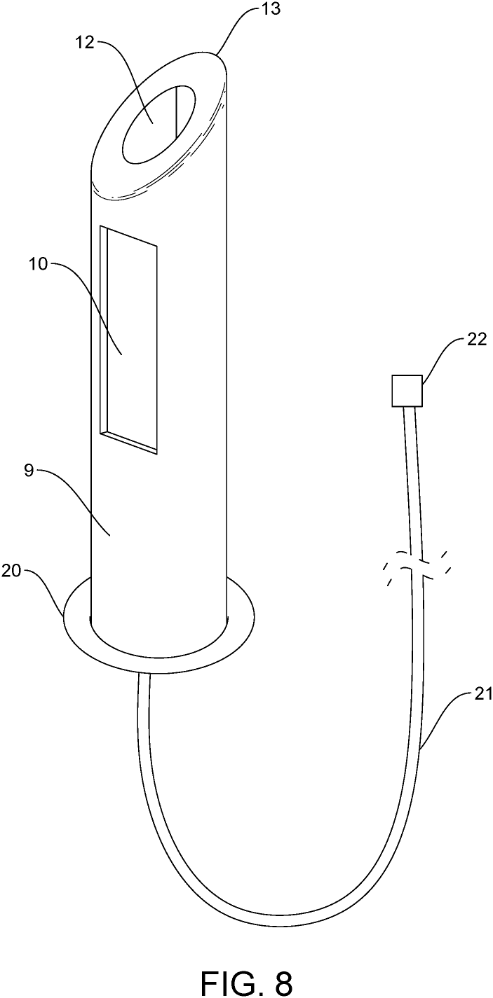

The image acquisition system is configured to transmit images captured by the camera (6) via the transmission line (21) that runs down the length of the sleeve of cover (9) to a wireless transmission device (24), the images are then wirelessly transmitted to a display device and shown on screen (27), as shown in FIG. 6A and FIG. 7. Wired transmission may also be used.

The attachment system of the VAD (5) to the TEE probe (1) is in the form of a “sleeve” (9) placed on the TEE probe (1). The sleeve (9) is a tubular, thin-flexible-elastic-walled, single, or multi-piece assembly, similar to the shape seen when looking from the outside of the TEE probe (1). This cover (9) may partially or completely cover the tip (3) of the TEE probe (1). There may be areas covered with adhesive material that partially or completely covers the inner surface of the cover (9).

The cover (9) can cover, leave open or partially cover the part with the sensor (2) on the TEE probe (1). Preferably, there is an opening (10) on the part of the cover (9) that comes over the sensor (2) so that the surface of the sensor (2) is completely free and unimpeded by the cover. This gap (10) on the cover (9) can also occur with the optional separation of an existing piece on the cover (9).

The VAD (5) completely covers the TEE probe shaft (4). The size of this cover (9) may initially be smaller, equal, or larger than the size of the TEE probe (1), or in combination, different parts of the cover (9) may be different sizes on the same cover.

In cases where the cover (9) is smaller, while the TEE probe (1) is placed on it, it can expand and grasp the TEE probe (1) owing to the flexible and elastic structure of the VAD (5). In this way, an elastic tensile force is created, and the VAD (5) can be firmly attached to the TEE probe (1).

In cases where the cover (9) is an equal or larger size than the TEE probe (1), the VAD (5) can be firmly attached to the TEE probe (1) and secured in place with the addition of external tension and/or compression forces. This tensile force can be an elastic material that can be wrapped around the VAD (5), or a system placed on the VAD (5) that reduces the diameter of the VAD (5) when the tensile force is applied from the outside, allowing it to attach to the TEE probe (1).

The materials that make up the VAD (5) are at least partially of elastic structure, which can expand when a force is applied, and of which shape thickness can change so that it adapts to the outer shape of the probe (1), can be firmly attached to the probe (1) and can move in all directions with the probe (1). The sleeve may incorporate non-elastic components, such as flexible non-elastic portions for sheathing the shaft of the TEE probe, or more rigid portions for stabilising attached electronics, but the portion sheathing the tip of the TEE probe is always elastic so as to maintain a firm grip on the probe and prevent slippage that would cause the sensors to become misaligned with the sleeve opening.

The diameter and dimensions of the VAD (5) before it is placed on the TEE probe (1) may be the same, smaller, or larger than the TEE probe (1). The material thickness of the cover may vary or may be equal at every point. In any part of the cover (9), there may be patterns formed partly by the thickening of the material. These patterns can be in any direction or symmetrical on the cover (9).

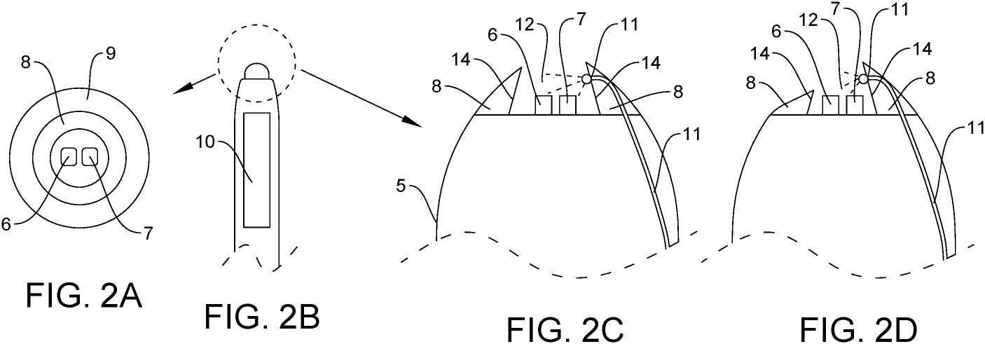

The camera (6) can take images and transmit these images via signals. As seen in FIGS. 2-A-B, this camera (6) can be positioned at different angles to the TEE probe sensor so that as the TEE probe (1) is moved towards the end (3) of the patient’s digestive tract the camera (6) can take images in the direction the probe is moving.

A light source (7) is located close to the location of the camera (6). This light source (7) can be a direct light producing source (LED etc.), or it can be a light source coming from a fiber optic line. The light source (7) can be positioned at angles to illuminate the direction the camera takes the image. The camera (6) and the light source (7) can be in multiple and variable positions with respect to one another.

The imaging system may have a light-permeable protective barrier (8). This barrier (8) may have features that are transparent, partially transparent, partially opaque, with a light filter, protecting the camera (6) and the light source (7) against impact and liquid contact. This barrier (8), which protects the camera (6) system, may consist of glass, plastic, plexiglass, acrylic, or material used in the camera and the optics industry, or a combination of these. The protective barrier (8) may consist of the same material as a whole or a continuation of the cover (9).

As shown in FIG. 2-C, in some cases, the protective barrier (8) is only located at the edges of the camera (6) and the light source (7), ensuring that the side from which the camera (6) takes images remains open. The barrier (8) located on the sides of the camera (6) and the light source (7) creates a potential protective gap (12) in the front of the camera (6). The protective barrier (8) can be of different sizes, angles, and inclinations around the camera (6) and the light source (7). The protective barrier (8) may cover the camera (6) and light source (7) by completely enclosing them or by only partially enclosing them.

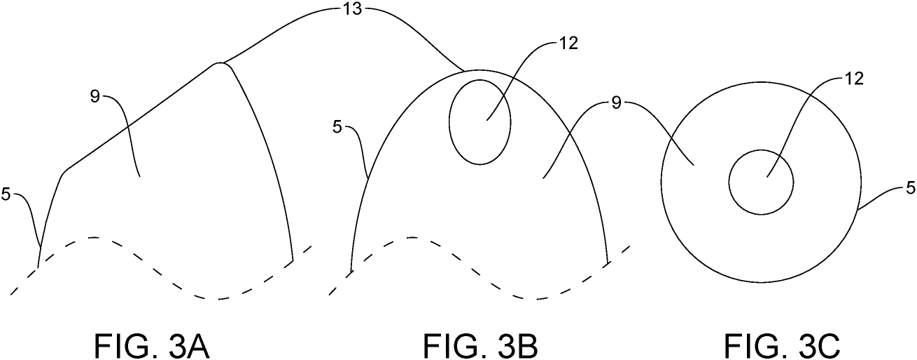

As can be seen from the side view in FIG. 3-A, the protective barrier (8) may in some examples make the tip of the VAD (5) take on a more curved and pointed shape in one direction (13). When viewed from the front in FIG. 3-B, it can be seen that the pointed part (13) of the protective barrier (8) is closer to the center. In this way, the edges (14) of the protective barrier (8) can be extended in different sizes (see FIG. 2C and FIG. 2D), and the tip-pointed part (13) of the VAD (5) can be positioned in different directions.

In the top view in FIG. 3-C, it can be seen that the edges (14) of the protective barrier (8) leave open the area of the protective gap (12) that the camera (6) displays. The edges (14) of the protective barrier (8) can form a protective gap (12) at varying angles and diameters in the direction the camera takes the image. This protective gap (12) may be one or more than one.

As seen in FIGS. 2-C-D, in another design, the protective edges (14) may include structures in the form of small pipes (11) for spraying liquid or air in the direction of the camera (6) and the light source (7). These pipes (11) follow the cover (9) all the way as part of the VAD (5). It carries the air or liquid injected from the outside towards the protective barrier edges (14). This liquid or air can be sprayed, if necessary, to clean the camera (6) and the light source (7).

In some examples, the injected air or fluid can inflate a balloon-shaped structure or space at the tip of the VAD (5) with the camera (6) (not shown in the pictures). These small pipes (11) allow liquid or air to be easily transmitted back and forth.

As seen in FIG. 7, the end part of these pipes outside the patient can be terminated to be compatible with an injector system. Pipes (11) can be one or more. The material forming these pipes may be the same material forming the VAD (5) or maybe different plastic, silicon, rubber, etc.

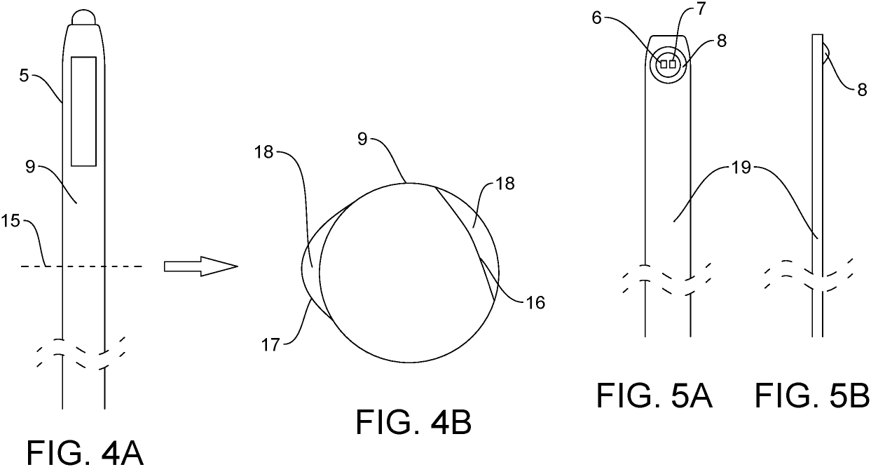

In FIG. 4-A, there is the point where the cross-section of VAD is taken, and in FIG. 4-B, there is the top view of the cross-section of the VAD. As can be understood from this section, there may be an inner layer (16) or an outer layer (17) on the inner or outer side of the elastic and flexible layer that forms one or more channels (18) in the cover (9) body of the VAD (5). These inner (16) and outer layers (17) may consist of more than one layer, either overlapping or side by side. There may be a channel (18) between these inner (16) and outer (17) layers and the cover (9), or alternatively they may be completely adhered to each other, or it may be initially closed and then open by a force.

Extra layers may cover part or all of the cover (9). These inner (16) and outer (17) layers may be on the inside or outside of the cover (9), contacting the TEE probe (1) shaft (4). These inner (16) and outer (17) layers extend across the VAD (5), creating potential gaps or channels (not shown in the illustrations). These channels may run over the VAD (5) to open into a gap or form a closed “blind” end.

Elements such as the transmission line (21) of the camera (6) and light source (7) system, a small pipe (11) line injecting liquid or air, and electrodes can pass through these channels. The channels can be sealed so that they can also be used to transmit liquids or air. The potential channels formed by these layered inners (16) and outer (17) layers can run on the VAD (5) in straight, spiral, or any other suitable geometry of path. Preferably, it is intended to provide a connection between the two ends of the VAD (5). E.g., transmission line (21), liquid-air injection, external device delivery. The materials that make up these inner (16) and outer (17) layers can be the same as the materials that make up the VAD (5), as well as different materials or a combination of more than one material.

As seen in FIGS. 5-A-B, the camera (6) and the light source (7) system can be constructed as separate parts from the cover (9) itself. This separate piece can then work together with the VAD (5) cover (9) body.

The camera (6), the light source (7) system, and the transmission line (21) that connects them with other main devices can be positioned on a flexible strip (19) like structure. This strip (19) can then be placed over the VAD (5) by advancing through the channels on the cover (9) of the VAD (5). The camera (6) and light source (7) systems can be located separately or simultaneously on a strip (19). The structure that carries the camera (6) and light source (7) systems is preferably in the form of a strip but can also be in the form of a wire or a pipe.

VAD (5) system elements can be seen in FIG. 6. As seen in FIG. 6-A, the cover (9) covering the TEE probe (1) shaft (4) of the VAD (5) can be folded over itself (20).

The exit of the transmission line (21) can be seen from the part of the VAD (5) cover (9) that contacts the TEE probe (1) shaft (4). The transmission line (21) can move freely on the surface of the VAD (5) cover (9) that contacts the TEE probe shaft (4), or it can be integrated with and adhered to the cover (9).

At the end of the transmission line (21), there is a connector (22) that provides the connection with the screen (27) of the wireless transmission device (24). The transmission line (21) can be flexible and bendable like the body of the VAD (5) cover (9).

In FIG. 6-B, the wireless transmission device (24) that transmits the images coming from the camera (6) wirelessly, processes the images, and manages the camera and light source is seen. The wireless transmission device (24) can send the signals coming from the camera (6) to other receiving devices, and the image and light settings can be configured through this device.

There is a connection point (25) on the device where the inputs for the camera (6) and the light source (7) can be connected to the transmission line (21). This port (25) may also include light source (7) system connections.

There is a gripping tool (26) compatible with the TEE probe (1) shaft (4) on the device - and which would also be compatible with shafts of different TEE probe designs. This gripping tool (26) may be flexible, a circle or a circle as a whole or a partial part. The gripping tool (26) may have different properties, such that it is firmly attached to the shaft (4) of the TEE probe (1). E.g., it may have magnet, Velcro, adhesive tape properties.

In FIG. 6-C, the display screen (27) on which the images coming from the VAD camera can be processed and viewed can be seen. This screen (27) may display images in-real time as the TEE probe is advanced through the digestive tract simultaneously, or with a pre-set delay. By touching the screen (27) or by pressing the keys, image and light settings, video recording and photo shooting can be done.

FIG. 7 shows the functioning of the VAD (5) system once installed on the TEE probe (1).

The VAD (5) cover (9) is placed over the TEE probe (1). The part with the camera (6) and the light source (7) is placed on the part of the TEE probe (1) with the sensor (2). The front part of the camera or the imaging direction is placed in such a way that it shows the direction in which the TEE probe (1) is advancing, preferably parallel to the long axis of the TEE probe (1).

When the part of the TEE probe (1) with the sensor (2) is activated by the operator, the camera (6) and the light source (7) move simultaneously as a part of the TEE probe (1). The cover (9) of the VAD (5) covers the the transmission line (21), and other connecting lines to create a smooth surface. The cover (9) can cover the shaft (4) of the TEE probe (1) by extending it to the desired size; this distance may vary according to the patient’s dimensions or the operator’s preference. The transmission line (21) and the small pipe connections (23) can be connected to the related devices such as catheter devices with fluid or air pumps, etc.

In another embodiment of the VAD (5), the light source (7) may be located further away from the portion of the VAD (5) with the camera. In this case, the light can be carried to the camera part of the VAD (5) with one or more fiber-optic lines. While one end of the fiber optic line is in the light source (7) external to the cover (9), the other end, which transmits the light, is in a position to illuminate the area where the camera (6) takes the image.

The materials that make up the part of the VAD (5) with elastic and flexible properties may consist of plastic, rubber, silicone, fabric, or materials used in the endoscopy industry. The cover (9) may also consist of only one of these materials or in combinations with each other. VAD (5) can be produced by shaping these materials through the formwork system.

The camera (6) and the light source (7) can be produced together, separately, and in more than one number. The camera (6) system can be any suitable camera system for use with the technologies used in this field, and has dimensions that can be positioned at the end of the TEE probe (1) where the sensor (2) is located. The electronic system of the camera (6) that creates the image can be found together with the camera (6), or the signals can be moved to a farther point from the camera, and the image can be processed.

The light source (7) can be at the end of the VAD (5) where the camera (6) is located, or the light produced at a different point can be carried here by a fiber optic line. A light source can be produced by combining the light source (7) and the fiber optic line. The fiber optic line can be found together with the transmission line (21), as a part of the cover (9), or independently and manufactured accordingly.

VAD (5) components: Camera (6) system, light source (7), fiber optic line, elastic cover (9), small pipes (11), transmission line (21), etc., can be combined in a mold as a single piece or in multiple molds. Or, these parts can be produced separately or as combinations of each other and later combined as a whole. Or they can be produced in accordance with the endoscope production technique. The wireless transmission device (24) and the screen (27) may be manufactured in accordance with a technique in the field.

It is noted that the visual assist device described above also provides an effective cover for medical probes to prevent transmission of infections, and that this benefit would be provided even without the presence of the visual assist apparatus.

Thus, the present disclosure further provides an anti-infection cover for a medical probe, the cover comprising a flexible sleeve, the sleeve being at least partially elastic, having a first open end for inserting a medical probe and a second closed end for encasing the tip of a medical probe, the second end comprising an opening for exposing a sensor of a medical probe housed therein. The cover may be provided with an antibacterial coating and may completely enclose the shaft of the medical probe installed therein to prevent transmission of infections.

The disclosed cover may share many structural similarities with the elastic sleeve of the VAD described in the previous embodiments, but without the need to include the electronics necessary for image capture.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the Visual Assistance Device have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A Visual Assistance Device (VAD), comprising:
an flexible sleeve, the sleeve being at least partially elastic, having a first open end for inserting an endoscopic device and a second closed end for encasing the tip of an endoscopic device, the second end comprising an opening for exposing a sensor of an endoscopic device housed therein;
an image capture device disposed at the tip the second end of the flexible sleeve and being oriented coaxially with the sleeve so as to capture images of a region in front of the second end of the sleeve;
one or more lighting elements positioned adjacent to the image capture device and being configured to illuminate an area about the image capture device; and
a control unit comprising a transceiver, the control unit being configured to receive image data from the image capture device and transmit the image data to one or more external devices, the control unit being positioned outside of the sleeve with a wired connection for image data transmission extending along the entire length of the sleeve interior from the image capture device to the control unit.

2. A Visual Assistance Device according to claim 1, wherein the flexible sleeve is formed of two or more layers of flexible material, including a first base layer that forms the interior wall and one or more secondary layers.

3. A Visual Assistance Device according to claim 2, wherein the one or more secondary layers are arranged to form one or more channels on the base layer.

4. A Visual Assistance Device according to claim 3, wherein the one or more channels are sealed to facilitate their use as a fluid conduit.

5. A Visual Assistance Device according to claim 3, wherein the image capture device and the one or more lighting elements are secured on a flexible strip which can be inserted through and removed from one of the channels to position the image capture unit in a desired position.

6. A Visual Assistance Device according to claim 3, wherein one or more of the channels has a fluidic pipe threaded through it, the pipe being coupled at the open end of the sleeve to an injector system.

7. A Visual Assistance Device according to claim 6, wherein the opposing opening of one or more of the fluidic pipes comprises a nozzle directed at the image capture unit and is thereby configured to spray clean the image capture unit while the VAD is in use.

8. A Visual Assistance Device according to claim 3, wherein the VAD further comprises an inflatable balloon membrane positioned at the second end of the sleeve.

9. A Visual Assistance Device according to claim 3, wherein the one or more channels house one or more fibre optic lines coupling the lighting elements to an external light source.

10. A Visual Assistance Device according to claim 1, further comprising a transparent protective cover arranged to at least partially cover the image capture device and lighting elements.

11. A Visual Assistance Device according to claim 10, wherein the protective cover is shaped into a pointed tip and oriented in a desired direction.

12. A Visual Assistance Device according to claim 1, wherein the control unit is configured to receive and transmit captured Image data in real-time.

13. A Visual Assistance Device according to claim 1, wherein the control unit is configured to receive and transmit captured Image data with a predetermined time delay.

14. A Visual Assistance Device according to claim 1, wherein the control unit comprises a gripping mechanism for securing itself to the shaft of an endoscopic device.

15. A Visual Assistance Device according to claim 1, wherein the control unit comprises an interface for adjusting one or more settings of the image capture device and the one or more lighting elements.

16. A Visual Assistance Device according to claim 1, further comprising a display device configured to communicate with the control unit to receive and display the captured image data.

17. A Visual Assistance Device according to claim 16, wherein there is a wired connection between the control unit and the display device.

18. A Visual Assistance Device according to claim 16, wherein the display device is configured to perform one or more image processing operations on the received image data.

19. A Visual Assistance Device according to claim 16, wherein the display device comprises a touchscreen interface and is configured to instruct the control unit to perform one or more operations.

20. A Visual Assistance Device according to claim 1, wherein the opening at the second end of the flexible sleeve is covered by a material configured to transmit ultrasonic waves.
